(19) United States
(12) Patent Application Publication
(10) Pub. No.: US 2025/0204953 A1
(43) Pub. Date: Jun. 26, 2025

(54) MULTI-PURPOSE SURGICAL PIN FOR FLUID DELIVERY

(71) Applicant: Matthew YOUNG, Madison, MS (US)

(72) Inventor: Matthew YOUNG, Madison, MS (US)

(21) Appl. No.: 18/987,236

(22) Filed: Dec. 19, 2024

Related U.S. Application Data
(60) Provisional application No. 63/614,232, filed on Dec. 22, 2023.

(51) Int. Cl. A61B 17/34; A61B 17/72

(52) U.S. Cl. CPC A61B 17/3472; A61B 17/7233

(57) ABSTRACT

Apparatus and techniques for delivering a fluid into an inner portion of a bone. An example pin for fluid delivery includes a pin having a distal end that is configured to be inserted into a bone. A proximal end of the pin is configured to support one or more sensors relative to the bone for a surgical procedure. The pin includes an inner bore extending from the proximal end of the pin. The inner bore is configured to house a rigid element that is removable from the inner bore. The pin includes one or more apertures extending into the inner bore. The inner bore is configured to receive a fluid at the proximal end of the pin, and the one or more apertures are configured to deliver the fluid to an inner portion of the bone.

### CROSS-REFERENCE TO RELATED APPLICATION(S)

The present Application for Patent claims priority to U.S. Provisional Application No. 63/614,232, filed Dec. 22, 2023, which is hereby expressly incorporated by reference herein in its entirety.

### FIELD OF THE DISCLOSURE

The present disclosure generally relates to medical devices and procedures, and more particularly, to apparatus and methods for fluid delivery to an inner portion of a bone.

### BACKGROUND

A prosthetic joint infection (PJI) is an infection involving a joint prosthesis (and adjacent tissue) which can occur in patients after a joint replacement procedure (or arthroplasty). Joint replacement procedures include total joint replacement procedures and partial joint replacement procedures. Common examples of joint replacement procedures include, but are not limited to, hip replacement procedures and knee replacement procedures. A joint replacement procedure may involve surgery for removal (total or partial) of the patient's joint and placement of an artificial joint (or prosthesis).

One risk associated with joint replacement procedures is that the patient develops a PJI. PJIs may occur several weeks after the joint replacement procedure or many years after the procedure. While PJIs are rare (e.g., only occurring after about one percent of joint replacement procedures), these infections are associated with increased patient pain and discomfort, high rates of revision surgery (replacement of the original joint prosthesis), prolonged hospital admission and rehabilitation times, decreased functional recovery, and/or substantial healthcare costs, which can burden both patients and healthcare systems/providers.

In order to prevent a PJI in a patient following a joint replacement procedure, antibiotics may be administered to the patient throughout the perioperative period. For example, antibiotics may be administered systemically to the patient before and during the joint replacement procedure. The patient may be instructed to take antibiotics as a preventive measure after the procedure, e.g., before a medical checkup. However, despite these precautions, the occurrence rate of PJIs remains about the same (e.g., at about one percent).

Improved systems and techniques for preventing PJIs are desirable in order to reduce the occurrence rate of PJIs.

### SUMMARY

Certain aspects of the present disclosure generally relate to apparatus and methods for fluid delivery to an inner portion of a bone as part of a medical procedure such as a joint replacement procedure.

Certain aspects of the present disclosure provide a pin. The pin includes a distal end and a proximal end. The distal end of the pin is configured to be inserted into a bone. The proximal end of the pin is configured to support one or more sensors relative to the bone for a surgical procedure. The pin generally includes an inner bore extending from the proximal end of the pin and one or more apertures extending into the inner bore. The inner bore is configured to house a rigid element that is removable from the inner bore. At the proximal end of the pin, the inner bore is configured to receive a fluid, and the one or more apertures are configured to deliver the fluid from the inner bore of the pin into an inner portion of the bone.

Certain aspects of the present disclosure provide a device. The device generally includes a pin and a rigid element. The pin generally includes a distal end, a proximal end, an inner bore extending from the proximal end of the pin, and one or more apertures extending from an outer diameter of the pin to the inner bore. The distal end of the pin is configured to be inserted into a bone. The rigid element is disposed in the inner bore of the pin, and the rigid element is removable from the inner bore of the pin. When the rigid element is removed from the inner bore, the pin is configured to deliver a fluid introduced at the proximal end of the pin through the inner bore and the one or more apertures of the pin to an inner portion of the bone. When the rigid element is disposed in the inner bore of the pin, the device is configured to support one or more sensors relative to the bone for a surgical procedure.

Certain aspects of the present disclosure provide a method. The method generally includes inserting a distal end of a pin into a bone, the pin being configured to support one or more sensors relative to the bone for a surgical procedure; removing a rigid element from an inner bore of the pin, the inner bore extending from a proximal end of the pin; injecting a fluid into the inner bore at the proximal end of the pin; and delivering the fluid to an inner portion of the bone via one or more apertures in the pin, the one or more apertures being in fluid communication with the inner bore of the pin.

Certain aspects of the present disclosure provide a method. The method generally includes actuating a distal end of a pin into a bone, the pin being configured to cause an aperture in the bone and being configured to support one or more sensors of a surgical system; injecting a fluid into an inner bore of the pin at a proximal end of the pin; delivering the fluid into an inner portion of the bone via one or more apertures of a portion of the pin disposed in the aperture of the bone, the one or more apertures being in fluid communication with the inner bore of the pin; and performing a surgical procedure using the surgical system while the pin supports the one or more sensors of the surgical system.

### BRIEF DESCRIPTION OF THE DRAWINGS

So that the manner in which the above-recited features of the present disclosure can be understood in detail, a more particular description, briefly summarized above, may be had by reference to aspects, some of which are illustrated in the appended drawings. It is to be noted, however, that the appended drawings illustrate only certain typical aspects of this disclosure and are therefore not to be considered limiting of its scope, for the description may admit to other equally effective aspects.

FIG. 1A illustrates a cross-sectional view of an example pin for fluid delivery with separable components, in accordance with certain aspects of the present disclosure.

FIG. 1B illustrates a cross-sectional view of the pin of FIG. 1A with the components combined, in accordance with certain aspects of the present disclosure.

FIG. 1C illustrates a top view of the pin of FIG. 1B, in accordance with certain aspects of the present disclosure.

FIG. 2 illustrates a front view of an example pin for fluid delivery, in accordance with certain aspects of the present disclosure.

FIG. 3A illustrates a representation of a pin inserted in a bone, in accordance with certain aspects of the present disclosure.

FIG. 3B illustrates a representation of a pin supporting one or more sensors of a surgical system, in accordance with certain aspects of the present disclosure.

FIG. 3C illustrates a representation of delivering fluid to an inner portion of a bone, in accordance with certain aspects of the present disclosure.

FIG. 4A illustrates a representation of multiple pins inserted in a bone, in accordance with certain aspects of the present disclosure.

FIG. 4B illustrates a representation of multiple pins supporting one or more sensors of a surgical system, in accordance with certain aspects of the present disclosure.

FIG. 4C illustrates a representation of delivering fluid to an inner portion of a bone, in accordance with certain aspects of the present disclosure.

FIG. 5 is a flow diagram depicting an example procedure, in accordance with certain aspects of the present disclosure.

FIG. 6 is a flow diagram depicting another example procedure, in accordance with certain aspects of the present disclosure.

To facilitate understanding, identical reference numerals have been used, where possible, to designate identical elements that are common to the figures. It is contemplated that elements disclosed in one aspect may be beneficially utilized on other aspects without specific recitation.

### DETAILED DESCRIPTION

Antibiotics may be administered systemically (e.g., orally, intravenously, etc.) to a patient before and during a joint replacement procedure in order to prevent a prosthetic joint infection (PJI) from occurring after the joint replacement procedure. Notably, antibiotics administered systemically may not produce sufficient target site concentrations of the antibiotics to prevent the PJI from occurring after the procedure. Thus, antibiotics may be injected, using a syringe, into an inner portion of a patient's bone to produce sufficient target site concentrations of the antibiotics in an effort to prevent the PJI from occurring.

Conventionally, a cannula (a thin, flexible tube inserted into the body to administer fluids) is connected to the syringe and then driven into the bone, by the syringe, such that a distal end of the cannula is disposed in the inner portion of the bone. The antibiotics are then delivered into the inner portion of the bone through the cannula via manipulation of a plunger of the syringe. However, delivering the antibiotics in this way causes additional trauma to the bone and leaves a dedicated, extraneous aperture in the bone, which may increase the risk of infection, fracturing, and/or other complications.

In order to overcome these limitations, certain aspects of the present disclosure include multi-purpose pins for delivering fluid. In addition to delivering fluid, these pins may also be used for another purpose during the joint replacement procedure, such as for supporting one or more sensors (e.g., of a computer navigation array). In certain aspects, a pin includes a distal end, a proximal end, and an inner bore extending from the proximal end. The inner bore is configured to house a removable rigid element, and the distal end of the pin is inserted into an inner portion of a bone while the rigid element is disposed in the inner bore.

In some examples, when the rigid element is disposed in the inner bore of the pin, the proximal end of the pin is configured to support one or more sensors of a surgical system for performing a joint replacement procedure. When the rigid element is removed from the inner bore of the pin, the inner bore is configured to receive a fluid (e.g., an antibiotic, such as vancomycin) at the proximal end of the pin. One or more apertures in the pin extend into the inner bore, and the one or more apertures are also disposed in the inner portion of the bone.

In certain aspects, the one or more apertures are in fluid communication with the inner bore, and the one or more apertures are configured to deliver the fluid into the inner portion of the bone. After the pin delivers the fluid into the inner portion of the bone, the rigid element may be replaced in the inner bore of the pin to support the one or more sensors of the surgical system and prevent the one or more sensors from actuating relative to the bone. A joint replacement procedure is then performed using the surgical system, without causing the additional trauma to the bone and without creating and leaving the additional aperture in the bone, as occurs in some conventional techniques described above.

Various aspects of the disclosure are described more fully hereinafter with reference to the accompanying drawings. This disclosure may, however, be embodied in many different forms and should not be construed as limited to any specific structure or function presented throughout this disclosure. Rather, these aspects are provided so that this disclosure will be thorough and complete, and will fully convey the scope of the disclosure to those skilled in the art. Based on the teachings herein, one skilled in the art should appreciate that the scope of the disclosure is intended to cover any aspect of the disclosure disclosed herein, whether implemented independently of or combined with any other aspect of the disclosure. For example, an apparatus may be implemented or a method may be practiced using any number of the aspects set forth herein. In addition, the scope of the disclosure is intended to cover such an apparatus or method which is practiced using other structure, functionality, or structure and functionality in addition to or other than the various aspects of the disclosure set forth herein. It should be understood that any aspect of the disclosure disclosed herein may be embodied by one or more elements of a claim.

Note that, as described herein, a distal end, segment, or portion of a component refers to the end, segment, or portion that is closer to a patient's body during use thereof (and further away from, for example, a user or operator). On the other hand, a proximal end, segment, or portion of the component refers to the end, segment, or portion that is distanced further away from the patient's body and is in proximity to, for example, a user or operator.

Note also that, as described herein, an inferior end, segment, or portion of a component refers to the end, segment, or portion that is beneath or lower such as a bottom or underside of a tissue or structure. Conversely, a superior end, segment, or portion of the component refers to the end, segment, or portion that is above or higher such as a top or topside of the tissue or structure.

### An Example Pin for Fluid Delivery

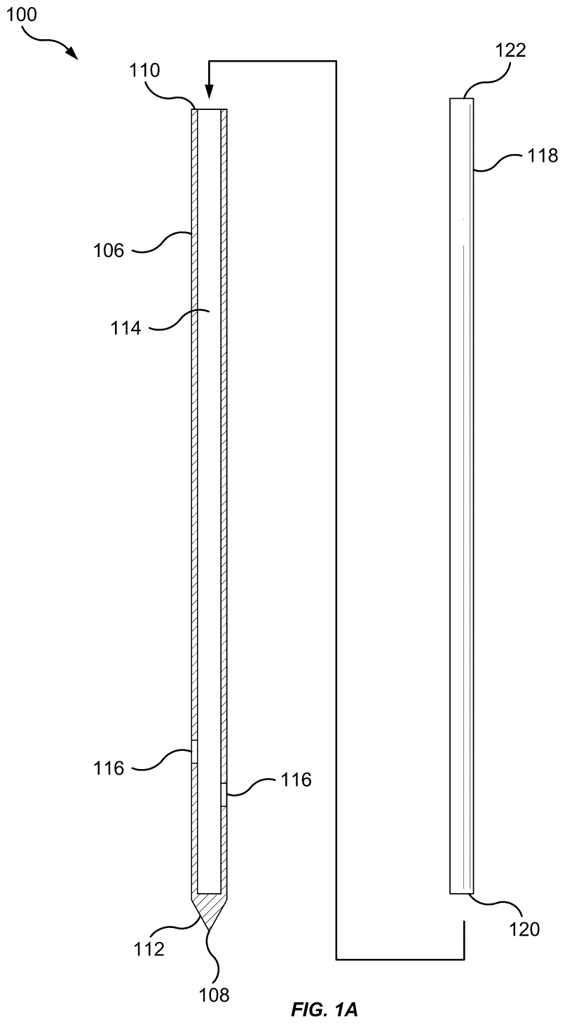

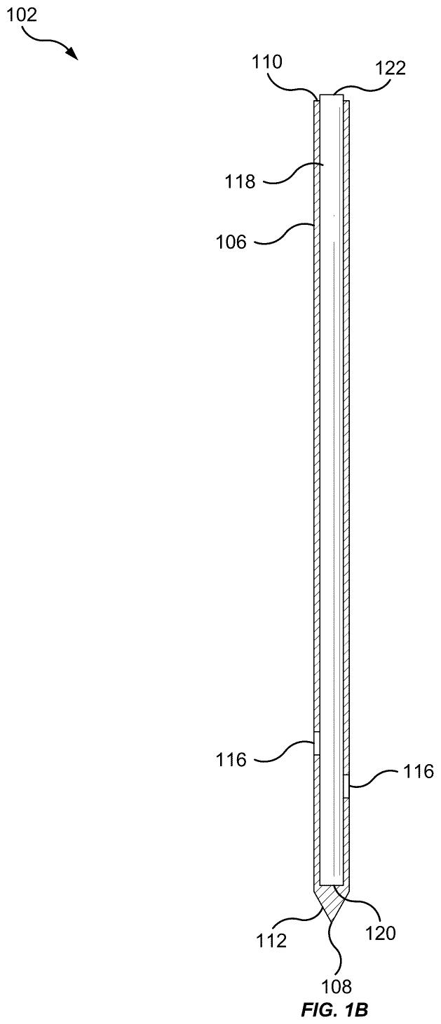

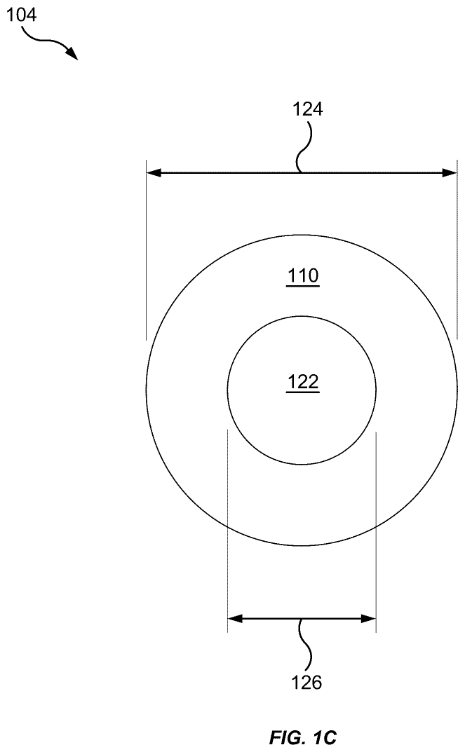

FIG. 1A illustrates a cross-sectional view 100 of an example pin for fluid delivery with separable components, in accordance with certain aspects of the present disclosure. FIG. 1B illustrates a cross-sectional view 102 of the pin for fluid delivery with the components combined, in accordance with certain aspects of the present disclosure. FIG. 1C illustrates a top view 104 of the pin for fluid delivery, in accordance with certain aspects of the present disclosure.

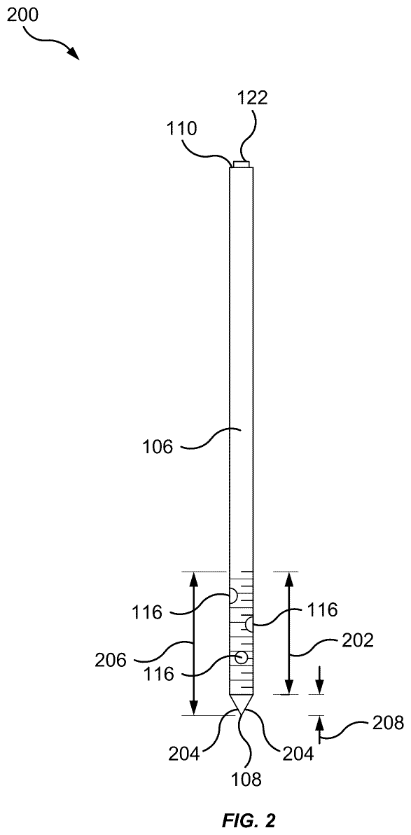

The pin 106 illustrated in FIGS. 1A-C and FIG. 2 may be capable of serving more than one purpose and may thus be referred to as a “multi-purpose pin.” For example, in some aspects, the pin 106 is configured to deliver fluid to an inner portion of a bone (e.g., as part of a joint replacement procedure), and in addition, the pin 106 may be configured to support one or more sensors (e.g., which may be used during the joint replacement procedure).

With reference to FIG. 1A, the cross-sectional view 100 illustrates a pin 106 having a distal end 108 and a proximal end 110. For instance, the pin 106 can include a tapered portion 112 which is illustrated as terminating at the distal end 108. The distal end 108 of the pin 106 is configured to be inserted into a bone (e.g., the tibia, femur, scapula, or humerus). In certain aspects, the tapered portion 112 may be configured to penetrate the bone, for example, during a joint replacement procedure. In certain aspects, the pin 106 may be configured for use on a smaller bone (e.g., a finger, toe, or other small bone). Such implementations of the pin 106 may be suitable for use in such procedures as treating open fractures, diabetic foot ulcers, or osteomyelitis, for example. The distal end 108 of the pin 106 may be configured to be removed from the bone, for example, after the joint replacement procedure.

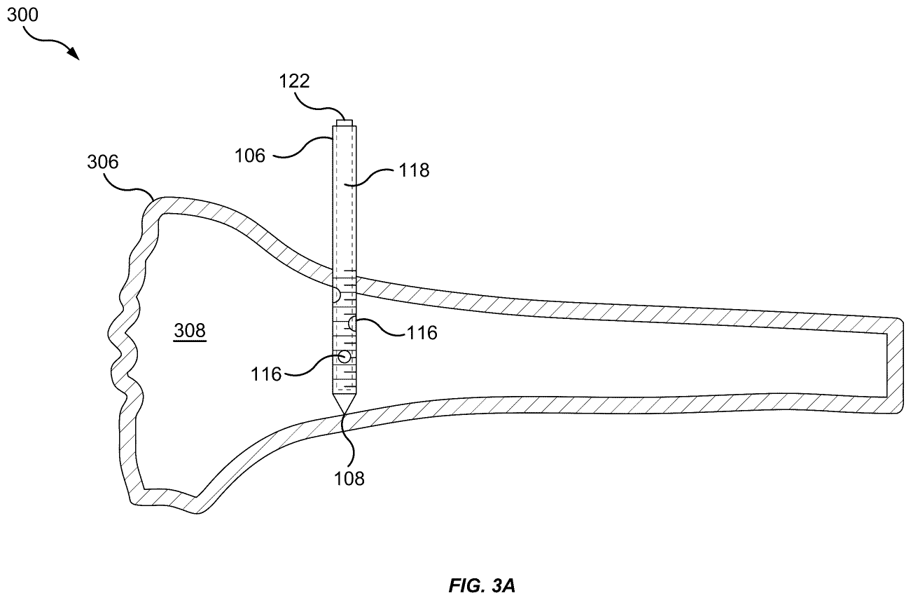

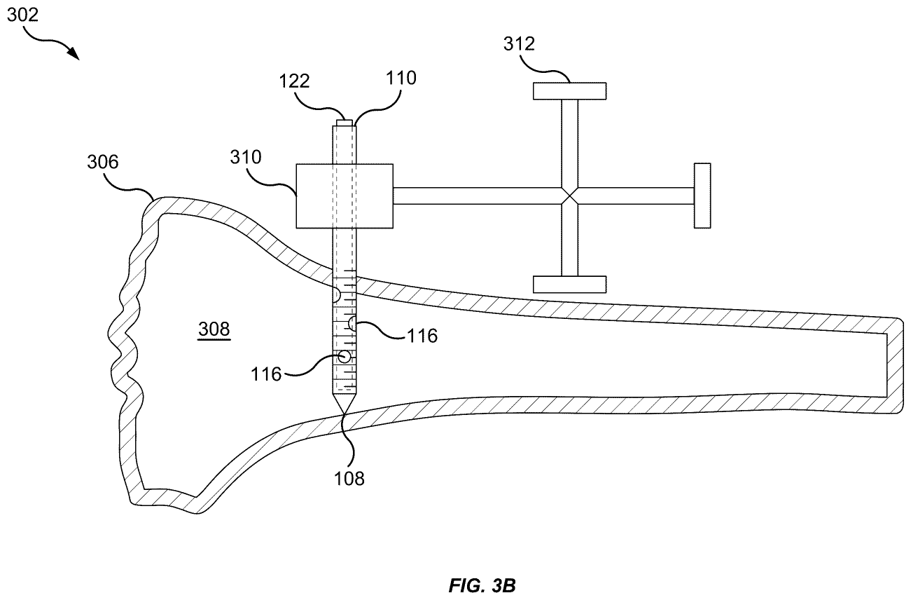

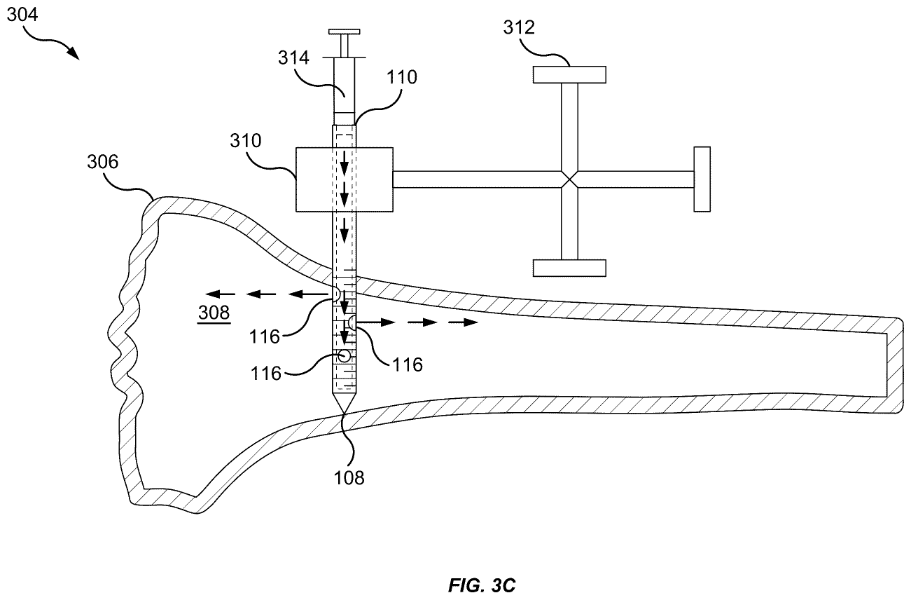

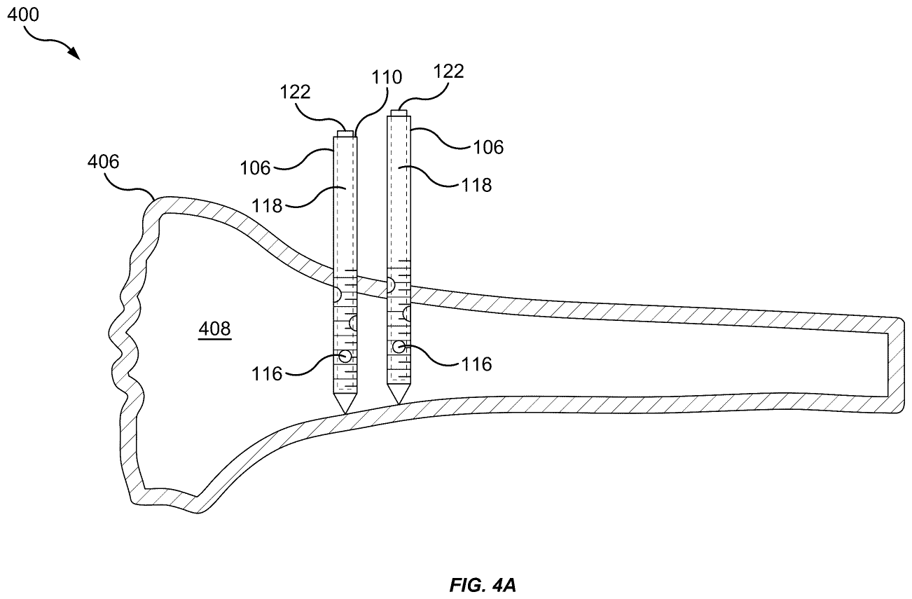

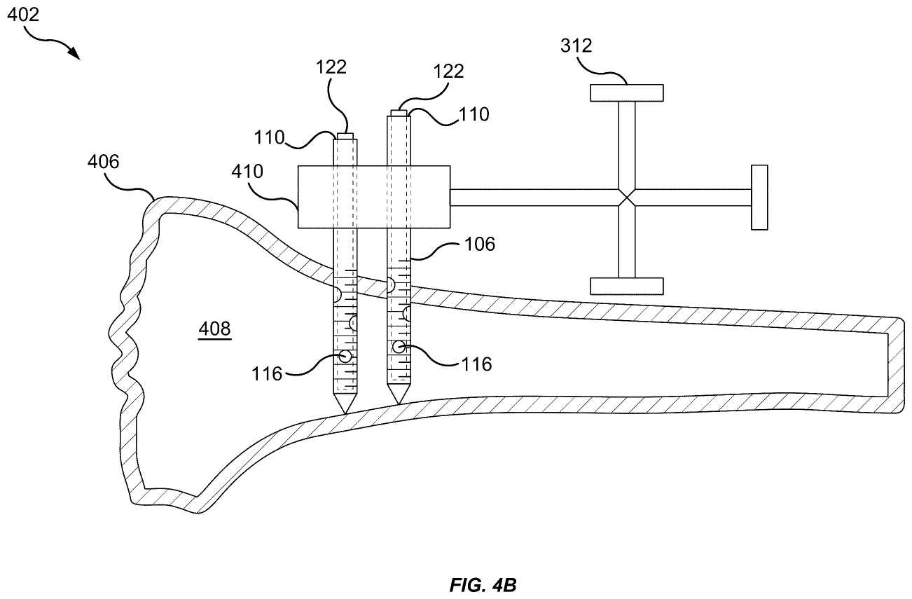

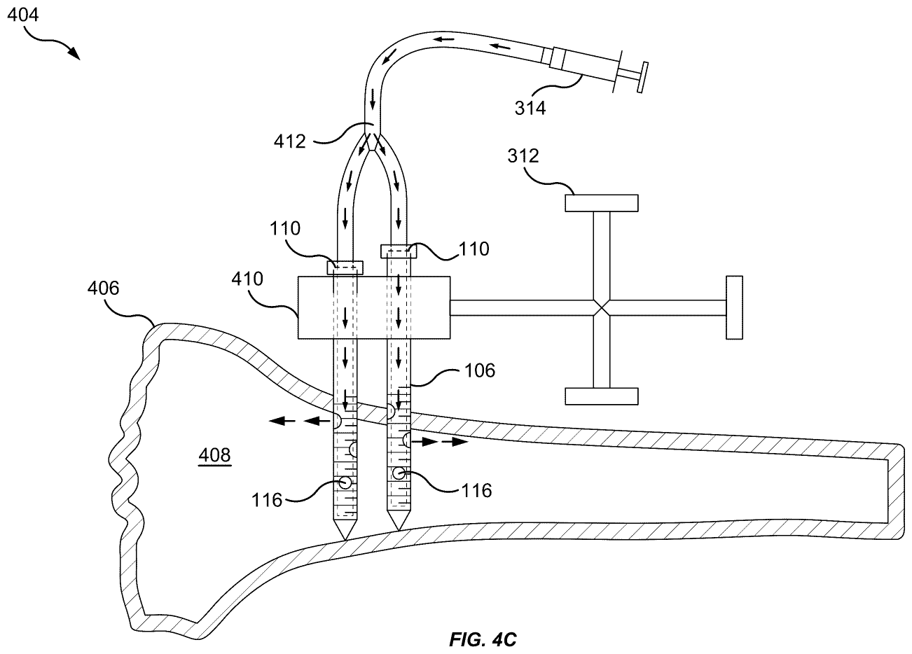

The proximal end 110 of the pin 106 can be configured to support one or more sensors relative to the bone for a surgical procedure as described in greater detail herein with respect to FIG. 3B and FIG. 4B.

The pin 106 includes an inner bore 114 extending from the proximal end 110 of the pin 106. In certain aspects, the inner bore 114 may be manufactured using a gun drilling process, extrusion, or any other suitable manufacturing process.

The pin 106 can include one or more apertures 116 extending from an outer surface of the pin to the inner bore 114. In certain aspects, the one or more apertures 116 can extend orthogonally to the inner bore 114 with respect to a longitudinal axis of the pin 106.

The inner bore 114 may be configured to receive a fluid (e.g., at the proximal end 110), and the one or more apertures 116 can be configured to deliver the fluid from the inner bore 114 into an inner portion of the bone, once the distal end 108 of the pin 106 is inserted into the bone, as described in greater detail with respect to FIG. 3C and FIG. 4C.

In certain aspects, the inner bore 114 is configured to house a rigid element 118. The rigid element 118 includes a distal end 120 and a proximal end 122. For instance, the rigid element 118 may be configured to be inserted and removed from the inner bore 114 as illustrated in the cross-sectional view 100 of FIG. 1A. In certain aspects, the rigid element 118 is a solid rod. In other aspects, the rigid element 118 is a cannula.

The rigid element 118 can be manufactured from the same material as the pin 106, or the rigid element 118 may be manufactured from a material that is different from the material of the pin 106. In certain aspects, the pin 106 and/or the rigid element 118 can be manufactured from a type of stainless steel suitable for medical applications (e.g., medical grade) such as super duplex stainless steel, duplex stainless steel, ferritic stainless steel, austenitic stainless steel, martensitic stainless steel, precipitation hardening stainless steel, etc. Alternatively, the pin 106 and/or the rigid element 118 may be composed of titanium (Ti) or any other suitable material. The pin 106 and/or the rigid element 118 may be manufactured from a material having a density which is high enough to be inserted into the bone without bending or deforming the pin 106 with the rigid element 118 inserted therein. In some aspects, the pin 106 is manufactured using a low-cost process and material, such that the pin 106 is configured to be disposable after the surgical procedure.

With reference to FIG. 1B, the cross-sectional view 102 depicts the rigid element 118 disposed in the inner bore 114 of the pin 106. In certain aspects, when the rigid element 118 is disposed in the inner bore 114, the proximal end 122 of the rigid element 118 extends a distance out from the inner bore 114 and the proximal end 110 of the pin 106. In some examples, a portion of the rigid element 118 which extends out from the proximal end 110 of the pin 106 when the rigid element 118 is disposed in the inner bore 114 may be configured to facilitate grasping or manipulation of the rigid element 118 in order to remove the rigid element 118 from the inner bore 114. For instance, the rigid element 118 can be grasped or manipulated using an operator's hand, forceps, a magnet, etc.

In certain aspects, the rigid element 118 can be disposed in the inner bore 114 before the distal end 108 of the pin 106 is inserted into the bone, where the rigid element 118 may prevent the pin 106 from bending or deforming during the insertion of the distal end 108 of the pin 106 into the bone. In one or more examples, the rigid element 118 may be disposed in the inner bore 114 when the proximal end 110 of the pin 106 (and/or the proximal end 122 of the rigid element 118) supports the one or more sensors relative to the bone for the surgical procedure as described in greater detail with respect to FIG. 3B and FIG. 4B. In certain aspects, dimensions of the rigid element 118 and dimensions of the inner bore 114 may be configured to prevent a press fit between the rigid element 118 and the inner bore 114 when the rigid element 118 is disposed in the inner bore 114.

With reference to FIG. 1C, the top view 104 illustrates the proximal end 110 of the pin 106 and the proximal end 122 of the rigid element 118. As shown, the pin 106 has an outer diameter 124, and the rigid element has an outer diameter 126. In the top view 104, the rigid element 118 is disposed in the inner bore 114 of the pin 106. In certain aspects, dimensions of the rigid element 118 and dimensions of the inner bore 114 are configured to minimize a distance between an outer diameter of the inner bore 114 and the outer diameter 126 of the rigid element 118 when the rigid element 118 is disposed in the inner bore 114. In some examples, when the rigid element 118 is disposed in the inner bore 114 of the pin 106, a distance between the outer diameter of the inner bore 114 and the outer diameter 126 of the rigid element 118 is less than 30 micrometers (μm), less than 20 micrometers, less than 10 micrometers, etc. That is, the diameter of the rigid element 118 may be configured to substantially fill the diameter of the inner bore 114.

The outer diameter 126 of the rigid element 118 is less than the outer diameter 124 of the pin 106. In certain aspects, the outer diameter 126 of the rigid element 118 has a first length, the outer diameter 124 of the pin 106 has a second length, and the first length may be less than 50 percent of the second length. In some examples, the outer diameter 124 of the pin 106 may be less than 4 millimeters (mm). In other examples, the outer diameter 124 of the pin 106 can be greater than or equal to 4 mm.

In certain aspects, the outer diameter 124 of the pin 106 has a length in a range of 2.9 to 4.1 millimeters (e.g., the outer diameter 124 may have a length of about 3.5 millimeters). In certain aspects, the outer diameter 126 of the rigid element 118 has a length in a range of 0.8 to 2.0 millimeters (e.g., the outer diameter 126 can have a length of about 1.4 millimeters). It is to be appreciated, however, that the outer diameter 124 may have a length of less than 2.9 millimeters or greater than 4.1 millimeters and the outer diameter 126 may have a length of less than 0.8 millimeters or greater than 2.0 millimeters.

FIG. 2 illustrates a front view 200 of an example pin for fluid delivery, in accordance with certain aspects of the present disclosure. In certain aspects, the pin 106 may include a threaded portion, which may be a male threaded portion 202 as illustrated in FIG. 2. In some examples, the male threaded portion 202 may be configured to secure the pin 106 within the bone such that actuations of the proximal end 110 of the pin 106 relative to the bone during a surgical procedure are prevented.

In certain aspects, the one or more apertures 116 may be disposed in the male threaded portion 202. However, in some examples, the one or more apertures 116 may be disposed outside of the male threaded portion 202 or both in and out of the male threaded portion 202. In certain aspects, the pin 106 may include a tapered portion 204 which terminates at the distal end 108 of the pin 106. Although a continuous taper is illustrated in the example pin 106 illustrated in FIG. 2, it is to be appreciated that the tapered portion 204 can include a variable taper, a bevel, etc. The tapered portion 204 may be symmetrical (as shown in FIG. 2) or asymmetrical (as illustrated in FIG. 1A) with respect to a longitudinal axis of the pin 106.

In various examples, the tapered portion 204 is configured to penetrate the bone such that the male threaded portion 202 may secure the pin 106 within the bone as the distal end 108 of the pin 106 is inserted into the bone and prevent the pin 106 from backing out of the bone. In certain aspects, the pin 106 includes a first length 206 which extends from a proximal end of the male threaded portion 202 to the distal end 108 of the pin 106. In some examples, the first length 206 is in a range of 30 to 70 millimeters (e.g., the first length 206 may be about 50 millimeters). In other examples, the first length 206 may be less than 30 millimeters or greater than 70 millimeters.

In certain aspects, the pin 106 includes a second length 208 which extends from a proximal end of the tapered portion 204 to the distal end 108 of the pin 106. In some examples, the second length 208 is in a range of 2 to 3 millimeters (e.g., the second length 208 may be a length of about 2.5 millimeters). In other examples, the second length 208 may be less than 2 millimeters or greater than 3 millimeters. In certain aspects, the second length 208 may be configured to penetrate a portion of the periosteum of the bone.

FIG. 3A illustrates a representation 300 of a multi-purpose pin inserted in a bone, in accordance with certain aspects of the present disclosure. FIG. 3B illustrates a representation 302 of the multi-purpose pin supporting one or more sensors of a surgical system, in accordance with certain aspects of the present disclosure. FIG. 3C illustrates a representation 304 of delivering fluid to an inner portion of a bone with the multi-purpose pin, in accordance with certain aspects of the present disclosure.

With reference to FIG. 3A, the representation 300 depicts a bone 306 and an inner portion 308 of the bone 306. In certain aspects, the bone 306 may be a tibia or a femur, for example. In other aspects, the bone 306 can be a scapula or a humerus. In some examples, the bone 306 is representative of many different bones, such as bones which may benefit from the apparatus and methods described herein. In certain aspects, the inner portion 308 of the bone 306 can be an intramedullary canal of the bone 306.

In FIG. 3A, the pin 106 is illustrated to be inserted in the bone 306 (with the rigid element 118 disposed in the inner bore 114 of the pin 106) such that the one or more apertures 116 are disposed in the inner portion 308 of the bone 306. The distal end 108 of the pin 106 may be inserted in a superior portion of the bone 306 while the rigid element 118 is disposed in the inner bore 114. In certain aspects, the pin 106 is driven through the bone 306 until the distal end 108 of the pin 106 is disposed in an inferior portion of the bone 306, as illustrated. In some examples, when the distal end 108 of the pin 106 is disposed in the inferior portion of the bone 306, the proximal end 110 of the pin 106 and the proximal end 122 of the rigid element 118 may extend out from the superior portion of the bone 306, as shown.

In the representation 302 depicted in FIG. 3B, the pin 106 is inserted in the bone 306 such that the proximal end 110 of the pin 106 and the proximal end 122 of the rigid element 118 extend out from the superior portion of the bone 306. In certain aspects, an alignment device 310 can be disposed over a portion of the pin 106 extending out from the superior portion of the bone 306 in order to support one or more sensors 312 of a surgical system. In some examples, the alignment device 310 is configured to couple the one or more sensors 312 to the pin 106 in order to perform a surgical procedure (e.g., a joint replacement procedure) using the one or more sensors 312.

In certain aspects, the one or more sensors 312 are part of an array of sensors for a computer navigation device of the surgical system. The array of sensors may also be referred to as a “computer navigation array.” In various examples, the sensors 312 are configured to facilitate navigation functionality of a surgical robot of the surgical system. In these examples, the pin 106, the rigid element 118, and the alignment device 310 may be configured to prevent actuations of the one or more sensors 312 relative to the bone 306. Notably, actuations of the one or more sensors 312 relative to the bone 306 may adversely affect the computer navigation device and/or the navigation functionality of the surgical robot.

With reference to FIG. 3C, the representation 304 includes a syringe 314 at least partially filled with a fluid. In certain aspects, the syringe 314 can be filled with an antibiotic, such as vancomycin. In certain aspects, the syringe 314 can be filled with any of various suitable pharmacologically active agents.

As illustrated in FIG. 3C, the rigid element 118 has been removed from the inner bore 114 of the pin 106 while the distal end 108 of the pin 106 is inserted in the bone 306. In certain aspects, when the rigid element 118 is removed from the inner bore 114 of the pin 106, the inner portion 308 of the bone 306 can be in fluid communication with the inner bore 114 via the one or more apertures 116.

In certain aspects, the syringe 314 is configured to inject the fluid into the inner bore 114 at the proximal end 110 of the pin 106. This injection may be performed directly into the inner bore or via tubing coupled between the syringe 314 and the proximal end 110 of the pin 106. In some examples, injecting the fluid into the inner bore 114 may be configured to deliver the fluid to the inner portion 308 of the bone 306 via the one or more apertures 116 of the pin 106. In one or more examples, delivering the fluid to the inner portion 308 of the bone 306 may be configured to prevent a prosthetic joint infection (PJI).

In certain aspects, an antibiotic administered systemically (e.g., orally, intravenously, etc.) may not produce sufficient target site concentrations of the antibiotic (e.g., within the inner portion 308 of the bone 306) to prevent a PJI from occurring after performing the surgical procedure. In some examples in which the fluid is an antibiotic, delivering the antibiotic to the inner portion 308 of the bone 306 can produce sufficient target site concentrations of the antibiotic to prevent the PJI from occurring (or at least reduce the risk of a PJI occurring) after performing the surgical procedure. Although it may be possible to produce sufficient target site concentrations of the antibiotic to prevent the PJI from occurring by, e.g., connecting a cannula to the syringe 314 and driving a distal end of the cannula through the bone 306 and into the inner portion 308 of the bone 306 to administer the antibiotic, this would cause additional trauma to the bone 306 and produce an additional aperture in the bone 306 (i.e., in addition to one or more apertures from pin(s) driven into the bone for supporting the sensor(s)). It is to be appreciated that the additional aperture in the bone 306 may increase a risk that the PJI occurs after performing the surgical procedure and may also increase risks of additional complications (e.g., fractures in the bone 306, recovery pain, etc.).

In certain aspects, by delivering the antibiotic to the inner portion 308 of the bone 306 through the inner bore 114 via the one or more apertures 116, the additional trauma and the additional aperture in the bone 306 are avoided. In some examples, the rigid element 118 may be disposed in the inner bore 114 of the pin 106 after delivering the antibiotic or other fluid to the inner portion 308 of the bone 306. In these examples, replacing the rigid element 118 in the inner bore 114 of the pin 106 can prevent actuations of the one or more sensors 312 relative to the bone 306.

FIG. 4A illustrates a representation 400 of multiple pins inserted in a bone (at least one of which may be a multi-purpose pin as described herein), in accordance with certain aspects of the present disclosure. FIG. 4B illustrates a representation 402 of the multiple pins supporting one or more sensors of a surgical system, in accordance with certain aspects of the present disclosure. FIG. 4C illustrates a representation 404 of delivering fluid to an inner portion of the bone, in accordance with certain aspects of the present disclosure.

The representation 400 of FIG. 4A illustrates a bone 406 and an inner portion 408 of the bone 406. In some examples, the bone 406 is a femur, a tibia, a humerus, a scapula, a clavicle, an ulna, a radius, a talus, a calcaneus, etc. In certain aspects, multiple pins 106 are inserted in the bone 406 such that the one or more apertures 116 of each of the pins 106 may be disposed in the inner portion 408 of the bone 406. For instance, the inner portion 408 may be an intramedullary canal of the bone 406.

With reference to FIG. 4B, the representation 402 includes an alignment device 410, which may be disposed over one or more of the pins 106. In certain aspects, the alignment device 410 is configured to couple the one or more sensors 312 of the surgical system to one or more of the pins 106 which are secured within the bone 406 (e.g., by the male threaded portion 202 of the pins 106). In some examples, the male threaded portion 202 of the pins 106 may be configured to prevent the proximal end 110 of the pins 106 from actuating relative to the bone 406. Since the alignment device 410 may couple the one or more sensors 312 to at least some of the pins 106, preventing the proximal end 110 of the pins 106 from actuating relative to the bone 406 can also prevent the one or more sensors 312 from actuating relative to the bone 406. In certain aspects, the alignment device 410 is adjusted (e.g., tightened) while the rigid elements 118 are disposed in the inner bores 114 of the pins 106 before delivering the fluid to the inner portion 408 of the bone 406.

The representation 404 of FIG. 4C includes the syringe 314 filled with the fluid and includes split tubing 412. As shown, the split tubing 412 may be a “Y tubing” which includes (i) one end configured to interface with the syringe 314 and (ii) two split ends that are each configured to interface with one of the pins 106 at the proximal end 110 of the pins 106. Although two split ends are illustrated, it is to be understood that the tubing may include more than two split ends, for example, for injecting fluid into more than two pins. As further shown, the rigid elements 118 are removed from the inner bore 114 of each of the pins 106.

In certain aspects, injecting the fluid (e.g., the antibiotic) out from the syringe 314 and into the split tubing 412 may be configured to cause the inner bore 114 of each of the pins 106 to receive the fluid at the proximal end 110 of the pins 106. In some examples, receiving the fluid in the inner bore 114 of each of the pins 106 may be configured to deliver the fluid to the inner portion 408 of the bone 406 via the one or more apertures 116 of the pins 106. In examples in which the fluid includes vancomycin, delivering the vancomycin to the inner portion 408 of the bone 406 can produce sufficient target site concentrations of the vancomycin to prevent the PJI from occurring after performing the surgical procedure.

In certain aspects, delivering the vancomycin to the inner portion 408 via the one or more apertures 116 of the pins 106 can prevent the PJI from occurring without causing additional trauma to the bone 406 or an additional aperture in the bone 406. This is not possible in scenarios in which the vancomycin is injected into the inner portion 408 of the bone 406 using the syringe 314 and a cannula (separate from the support pins, such as pins 106) inserted in the bone 406. In some examples, after delivering the vancomycin to the inner portion 408 of the bone 406, the split tubing 412 is removed from the proximal end 110 of the pins 106, and the rigid elements 118 are replaced in the inner bore 114 of the pins 106.

In some aspects, while the one or more sensors 312 (e.g., of the computer navigation array) are shown extending generally horizontally (with respect to the longitudinal axis of the bone 306 and the bone 406) from the alignment device 310 or the alignment device 410, in some implementations the one or more sensors 312 may extend vertically or diagonally from the alignment device 310 or the alignment device 410.

### Example Procedures Using Multi-Purpose Pins

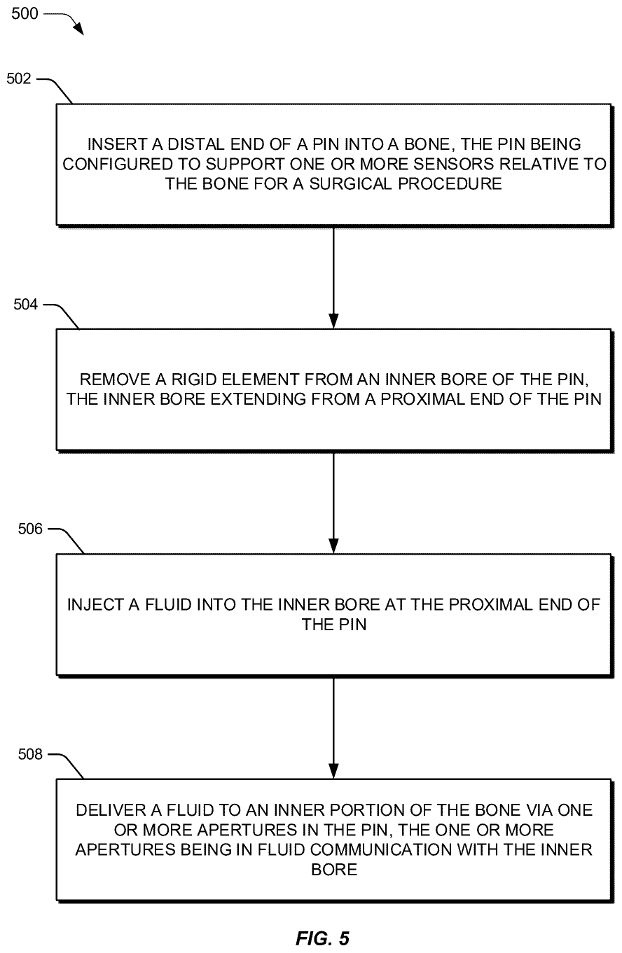

FIG. 5 is a flow diagram depicting a first procedure 500, in accordance with certain aspects of the present disclosure. The first procedure 500 may be performed or controlled, for example, by a human operator and/or a robot.

The first procedure 500 may begin, at block 502, with a distal end of a pin (e.g., distal end 108 of pin 106) being inserted into a bone (e.g., bone 306, 406). The pin may be configured to support one or more sensors (e.g., sensor(s) 312) relative to the bone for a surgical procedure. In some examples, the pin is configured to support the one or more sensors relative to the bone for a joint replacement procedure.

At block 504, a rigid element (e.g., rigid element 118) is removed from an inner bore (e.g., inner bore 114) of the pin. In some examples, the inner bore extends from a proximal end of the pin (e.g., proximal end 110 of pin 106).

At block 506, a fluid is injected into the inner bore at the proximal end of the pin. In some examples, the fluid includes an antibiotic (e.g., vancomycin), the fluid is a pharmacologically active agent, etc.

At block 508, the fluid is delivered to an inner portion (e.g., inner portion 308, 408) of the bone via one or more apertures (e.g., apertures 116) in the pin. The one or more apertures are in fluid communication with the inner bore.

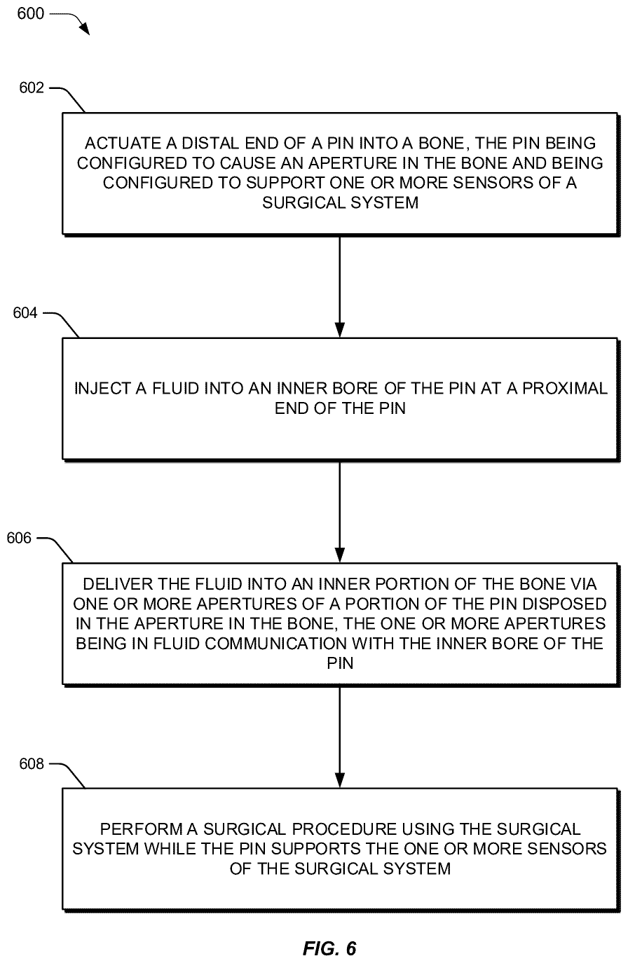

FIG. 6 is a flow diagram depicting a second procedure 600, in accordance with certain aspects of the present disclosure. The second procedure 600 may be performed or controlled, for example, by a human operator (e.g., a surgeon) and/or a robot.

The second procedure may begin, at block 602, with a distal end of a pin (e.g., the distal end 108 of pin 106) being actuated into a bone (e.g., bone 306, 406). The pin may be configured to cause an aperture in the bone and may be configured to support one or more sensors (e.g., sensor(s) 312) of a surgical system.

At block 604, a fluid is injected into an inner bore (e.g., inner bore 114) of the pin at a proximal end (e.g., proximal end 110) of the pin. In some examples, the fluid includes an antibiotic, such as vancomycin, or another pharmacologically active agent.

At block 606, the fluid is delivered into an inner portion (e.g., inner portion 308, 408) of the bone via one or more apertures (e.g., apertures 116) of a portion of the pin (e.g., the first length 206) disposed in the aperture in the bone. The one or more apertures are in fluid communication with the inner bore of the pin.

At block 608, a surgical procedure is performed using the surgical system while the pin supports the one or more sensors of the surgical system. In certain aspects, the surgical procedure is a joint replacement procedure. In some examples, the joint replacement procedure is performed using the computer navigation and/or the navigation functionality of the surgical robot.

According to certain aspects, the second procedure 600 further involve (i) disposing a rigid element (e.g., rigid element 118) in the inner bore of the pin before actuating the distal end of the pin into the bone and (ii) removing the rigid element from the pin before injecting the fluid into the inner bore of the pin. In certain aspects, the second procedure 600 further includes disposing the rigid element in the inner bore of the pin before performing the surgical procedure, such that the rigid element is disposed in the pin during the surgical procedure.

### Example Aspects

In addition to the various aspects described above, specific combinations of aspects are within the scope of the disclosure, some of which are detailed below:

Aspect 1: A pin comprising: a distal end, the distal end of the pin being insertable into a bone; a proximal end, the proximal end of the pin being configured to support one or more sensors relative to the bone for a surgical procedure; an inner bore extending from the proximal end of the pin, the inner bore being configured to house a rigid element that is removable from the inner bore; and one or more apertures extending into the inner bore, the inner bore at the proximal end of the pin being configured to receive a fluid and the one or more apertures being configured to deliver the fluid from the inner bore of the pin into an inner portion of the bone.

Aspect 2: The pin of Aspect 1, wherein the fluid comprises an antibiotic.

Aspect 3: The pin of Aspect 2, wherein the antibiotic comprises vancomycin.

Aspect 4: The pin of any combination of Aspects 1-3, wherein the fluid comprises a pharmacologically active agent.

Aspect 5: The pin of any combination of Aspects 1-4, further comprising a tapered portion terminating at the distal end of the pin, the tapered portion being configured to penetrate the bone.

Aspect 6: The pin of any combination of Aspects 1-5, further comprising a male threaded portion configured to secure the pin within the bone.

Aspect 7: The pin of any combination of Aspects 1-6, wherein an outer diameter of the pin is less than 4 millimeters.

Aspect 8: The pin of any combination of Aspects 1-7, wherein the bone is a tibia or a femur.

Aspect 9: The pin of any combination of Aspects 1-8, wherein the distal end of the pin is removable from the bone.

Aspect 10: The pin of any combination of Aspects 1-9, wherein the rigid element includes a solid rod.

Aspect 11: The pin of any combination of Aspects 1-10, wherein the rigid element comprises a cannula.

Aspect 12: The pin of any combination of Aspects 1-11, wherein an outer diameter of the rigid element has a first length, wherein an outer diameter of the pin has a second length, and wherein the first length is less than 50 percent of the second length.

Aspect 13: The pin of any combination of Aspects 1-12, wherein the inner portion of the bone comprises an intramedullary canal of the bone.

Aspect 14: The pin of any combination of Aspects 1-13, wherein the one or more apertures extend orthogonally into the inner bore with respect to a longitudinal axis of the pin.

Aspect 15: A device comprising: a pin having a distal end, a proximal end, an inner bore extending from the proximal end of the pin, and one or more apertures extending from an outer diameter of the pin to the inner bore, wherein the distal end of the pin is configured to be inserted into a bone; and a rigid element disposed in the inner bore of the pin, the rigid element being removable from the inner bore of the pin, wherein: when the rigid element is removed from the inner bore, the pin is configured to deliver a fluid introduced at the proximal end of the pin through the inner bore and the one or more apertures of the pin to an inner portion of the bone; and when the rigid element is disposed in the inner bore of the pin, the device is configured to support one or more sensors relative to the bone for a surgical procedure.

Aspect 16: The device of Aspect 15, wherein the bone is a tibia or a femur.

Aspect 17: The device of Aspect 15 or 16, wherein the fluid comprises an antibiotic.

Aspect 18: The device of Aspect 17, wherein the antibiotic comprises vancomycin.

Aspect 19: The device of any combination of Aspects 15-18, wherein the fluid comprises a pharmacologically active agent.

Aspect 20: The device of any combination of Aspects 15-19, wherein the distal end of the pin is configured to be removed from the bone.

Aspect 21: The device of any combination of Aspects 15-20, wherein the rigid element includes a solid rod.

Aspect 22: The device of any combination of Aspects 15-20, wherein the rigid element comprises a cannula.

Aspect 23: The device of any combination of Aspects 15-22, wherein the pin further comprises a male threaded portion configured to prevent an actuation of the proximal end relative to the bone.

Aspect 24: The device of any combination of Aspects 15-23, wherein the pin further comprises a tapered portion of the pin configured to penetrate the bone.

Aspect 25: The device of any combination of Aspects 15-24, wherein the inner portion of the bone comprises an intramedullary canal of the bone.

Aspect 26: The device of any combination of Aspects 15-25, wherein the one or more apertures extend orthogonally into the inner bore with respect to a longitudinal axis of the pin.

Aspect 27: The device of any combination of Aspects 15-26, wherein the device is configured to be inserted into the bone when the rigid element is disposed in the inner bore of the pin.

Aspect 28: A method of medical surgery, comprising: inserting a distal end of a pin into a bone, the pin being configured to support one or more sensors relative to the bone for a surgical procedure; removing a rigid element from an inner bore of the pin, the inner bore extending from a proximal end of the pin; injecting a fluid into the inner bore at the proximal end of the pin; and delivering the fluid to an inner portion of the bone via one or more apertures in the pin, the one or more apertures being in fluid communication with the inner bore of the pin.

Aspect 29: The method of Aspect 28, further comprising disposing the rigid element in the inner bore before inserting the distal end of the pin into the bone.

Aspect 30: The method of Aspect 28 or 29, further comprising replacing the rigid element in the inner bore of the pin after delivering the fluid.

Aspect 31: The method of any combination of Aspects 28-30, wherein the fluid comprises an antibiotic.

Aspect 32: The method of Aspect 31, wherein the antibiotic comprises vancomycin.

Aspect 33: The method of any combination of Aspects 28-32, wherein the fluid comprises a pharmacologically active agent.

Aspect 34: The method of any combination of Aspects 28-33, wherein the bone is a tibia or a femur.

Aspect 35: The method of any combination of Aspects 28-33, wherein the bone is a scapula or a humerus.

Aspect 36: The method of any combination of Aspects 28-35, wherein the one or more sensors comprise an array of sensors for a computer navigation device.

Aspect 37: The method of any combination of Aspects 28-36, further comprising: coupling the one or more sensors to the pin; and performing the surgical procedure using the one or more sensors.

Aspect 38: The method of any combination of Aspects 28-37, wherein the rigid element comprises a cannula.

Aspect 39: The method of any combination of Aspects 28-38, wherein the inner portion of the bone comprises an intramedullary canal of the bone.

Aspect 40: The method of any combination of Aspects 28-39, wherein the one or more apertures extend orthogonally into the inner bore with respect to a longitudinal axis of the pin.

Aspect 41: A method of medical surgery, comprising: actuating a distal end of a pin into a bone, the pin being configured to cause an aperture in the bone and being configured to support one or more sensors of a surgical system; injecting a fluid into an inner bore of the pin at a proximal end of the pin; delivering the fluid into an inner portion of the bone via one or more apertures of a portion of the pin disposed in the aperture in the bone, the one or more apertures being in fluid communication with the inner bore of the pin; and performing a surgical procedure using the surgical system while the pin supports the one or more sensors of the surgical system.

Aspect 42: The method of Aspect 41, further comprising: disposing a rigid element in the inner bore of the pin before actuating the distal end of the pin into the bone; and removing the rigid element from the pin before injecting the fluid into the inner bore of the pin.

Aspect 43: The method of Aspect 42, further comprising disposing the rigid element in the inner bore of the pin before performing the surgical procedure, such that the rigid element is disposed in the pin during the surgical procedure.

### Additional Considerations

The above-disclosed subject matter is to be considered illustrative, and not restrictive, and the appended claims are intended to cover all such modifications, enhancements, and other aspects which fall within the true spirit and scope of the present disclosure. Thus, to the maximum extent allowed by law, the scope of the present disclosure is to be determined by the broadest permissible interpretation of the following claims and their equivalents, and shall not be restricted or limited by the foregoing detailed description.

1. A pin comprising:
a distal end, the distal end of the pin being insertable into a bone;
a proximal end, the proximal end of the pin being configured to support one or more sensors relative to the bone for a surgical procedure;
an inner bore extending from the proximal end of the pin, the inner bore being configured to house a rigid element that is removable from the inner bore; and
one or more apertures extending into the inner bore, the inner bore at the proximal end of the pin being configured to receive a fluid and the one or more apertures being configured to deliver the fluid from the inner bore of the pin into an inner portion of the bone.

2. The pin of claim 1, further comprising a tapered portion terminating at the distal end of the pin, the tapered portion being configured to penetrate the bone.

3. The pin of claim 1, further comprising a male threaded portion configured to secure the pin within the bone.

4. The pin of claim 1, wherein an outer diameter of the pin is less than 4 millimeters.

5. The pin of claim 1, wherein the distal end of the pin is removable from the bone.

6. The pin of claim 1, wherein the one or more apertures extend orthogonally into the inner bore with respect to a longitudinal axis of the pin.

7. A device comprising:
a pin having a distal end, a proximal end, an inner bore extending from the proximal end of the pin, and one or more apertures extending from an outer diameter of the pin to the inner bore, wherein the distal end of the pin is configured to be inserted into a bone; and
a rigid element disposed in the inner bore of the pin, the rigid element being removable from the inner bore of the pin, wherein:
when the rigid element is removed from the inner bore, the pin is configured to deliver a fluid introduced at the proximal end of the pin through the inner bore and the one or more apertures of the pin to an inner portion of the bone; and
when the rigid element is disposed in the inner bore of the pin, the device is configured to support one or more sensors relative to the bone for a surgical procedure.

8. The device of claim 7, wherein the rigid element includes a solid rod.

9. The device of claim 7, wherein the rigid element comprises a cannula.

10. The device of claim 7, wherein the pin further comprises a male threaded portion configured to prevent an actuation of the proximal end relative to the bone.

11. The device of claim 7, wherein the pin further comprises a tapered portion configured to penetrate the bone.

12. The device of claim 7 wherein the one or more apertures extend orthogonally into the inner bore with respect to a longitudinal axis of the pin.

13. The device of claim 7, wherein the device is configured to be inserted into the bone when the rigid element is disposed in the inner bore of the pin.

14. The device of claim 7, wherein an outer diameter of the rigid element has a first length, wherein an outer diameter of the pin has a second length, and wherein the first length is less than 50 percent of the second length.

15. A method of medical surgery, comprising:
inserting a distal end of a pin into a bone, the pin being configured to support one or more sensors relative to the bone for a surgical procedure;
removing a rigid element from an inner bore of the pin, the inner bore extending from a proximal end of the pin;
injecting a fluid into the inner bore at the proximal end of the pin; and
delivering the fluid to an inner portion of the bone via one or more apertures in the pin, the one or more apertures being in fluid communication with the inner bore of the pin.

16. The method of claim 15, further comprising disposing the rigid element in the inner bore before inserting the distal end of the pin into the bone.

17. The method of claim 15, further comprising replacing the rigid element in the inner bore of the pin after delivering the fluid.

18. The method of claim 15, wherein the fluid comprises an antibiotic and wherein the antibiotic is vancomycin.

19. The method of claim 15, wherein the bone is a tibia, a femur, a scapula, or a humerus.

20. The method of claim 15, wherein the one or more sensors comprise an array of sensors for a computer navigation device.

21. The method of claim 15, further comprising:
coupling the one or more sensors to the pin; and
performing the surgical procedure using the one or more sensors.

22. The method of claim 15, wherein the inner portion of the bone comprises an intramedullary canal of the bone.

23. A method of medical surgery, comprising:
actuating a distal end of a pin into a bone, the pin being configured to cause an aperture in the bone and being configured to support one or more sensors of a surgical system;
injecting, at a proximal end of the pin, a fluid into an inner bore of the pin;
delivering the fluid into an inner portion of the bone via one or more apertures of a portion of the pin disposed in the aperture in the bone, the one or more apertures being in fluid communication with the inner bore of the pin; and
performing a surgical procedure using the surgical system while the pin supports the one or more sensors of the surgical system.

24. The method of claim 23, further comprising:
disposing a rigid element in the inner bore of the pin before actuating the distal end of the pin into the bone; and
removing the rigid element from the pin before injecting the fluid into the inner bore of the pin.

25. The method of claim 24, further comprising disposing the rigid element in the inner bore of the pin before performing the surgical procedure, such that the rigid element is disposed in the pin during the surgical procedure.
